(19) United States
(12) Patent Application Publication
(10) Pub. No.: US 2014/0065634 A1
(43) Pub. Date: Mar. 6, 2014

(54) CHIMERIC ANTI-dsDNA/CHROMATIN ANTIBODY

(71) Applicant: Bio-Rad Laboratories, Inc., Hercules, CA (US)

(72) Inventors: Roger Walker, Benicia, CA (US); John Wesley Breneman, III, Brentwood, CA (US)

(73) Assignee: Bio-Rad Laboratories, Inc., Hercules, CA (US)

(21) Appl. No.: 14/019,150

(22) Filed: Sep. 5, 2013

Related U.S. Application Data
(60) Provisional application No. 61/696,894, filed on Sep. 5, 2012.

(51) Int. Cl. C12Q 1/68

(52) U.S. Cl. CPC C12Q 1/6804
USPC 435/6.19; 530/387.3; 530/391.3

(57) ABSTRACT

Provided herein are antibodies for determining the concentration of anti-dsDNA and anti-chromatin antibodies in biological samples.

### CROSS-REFERENCE TO RELATED APPLICATIONS

The present disclosure claims benefit of priority to U.S. Provisional Patent Application No. 61/696,894, filed Sep. 5, 2012, which is incorporated by reference for all purposes.

### BACKGROUND OF THE INVENTION

Systemic autoimmune disorders are characterized by circulating autoantibodies to certain antigens, such as those present in the cell nucleus. Antinuclear antibodies (ANAs) can also be indicative of viral and bacterial infections, hypertension, cancers, and psoriasis.

The presence of autoantibodies, including ANAs, in a patient sample can be determined using immunoassays that typically rely on contacting the patient sample with the antigen, and determining whether the autoantibody is present using a labeled detection agent such as a secondary antibody, Protein A, Protein G, etc.

While detection of autoantibodies can be straightforward, difficulties arise in determining the relative amount of the autoantibody in the sample. Autoantibodies and ANAs are naturally present in the bloodstream, though usually at a low level. Thus determining the relative level or concentration of a particular ANA can avoid a false positive diagnosis, or a misdiagnosis. Such a determination requires a control or calibration antibody specific for the same antigen, that can be used to produce a calibration curve indicating the signal produced by various, known amounts of the calibration antibody. The signal from the patient sample can then be compared to the calibration curve to determine the relative amount of autoantibody in the patient sample.

Antibodies that can be used for accurate calibration, however, are not readily available. Ideally, the calibration antibody is similar to the native autoantibody to be detected so that assay conditions are kept as constant as possible, and the concentration determination is accurate. For this reason, present methods often utilize autoantibodies from native sources. Such autoantibodies, however, do not have predictable properties and are rare, so that obtaining a reliable source of calibration antibody is difficult and costly.

Disclosed herein are antibodies (e.g., chimeric antibodies or single chain antibodies) that bind to the same autoantigens targeted by autoantibodies found circulating in persons with autoimmune disorders. The presently disclosed antibodies have similar properties as the native autoantibodies to be detected, and are easily and predictably produced.

### BRIEF SUMMARY OF THE INVENTION

Provided herein are antibodies for use as standards for detection and/or characterization of anti-dsDNA or anti-chromatin antibodies. In some embodiments, the presently described antibodies are single chain antibodies (e.g., scFv) that specifically bind double-stranded DNA (dsDNA) and chromatin. In some embodiments, the presently described antibodies are chimeric, and specifically bind double-stranded DNA (dsDNA) and chromatin. Such antibodies are referred to as chimeric anti-dsDNA/chromatin antibodies. In some embodiments, at least part of the constant region of the chimeric antibody is derived from a human antibody, e.g., a part of the constant region specifically recognized by a secondary antibody, Protein A, Protein G, or Protein A/G. In some embodiments, the constant region is derived from a human antibody. In some embodiments, the constant region and framework regions are derived from a human antibody. The antibody isotype can be IgG (IgG1, IgG2, IgG3, IgG4), IgM, IgA, IgE, or IgD. In some embodiments, the chimeric antibody comprises complementarity determining regions (CDRs) derived from a non-human animal. In some embodiments, chimeric antibody comprises a variable region derived from a non-human animal. In some embodiments, the non-human animal is selected from a rodent (mouse, rat, hamster), rabbit, horse, goat, pig, sheep, chicken, and bovine.

In some embodiments, the anti-dsDNA/chromatin antibody is stable at 5° C. for at least 5 months, e.g., at least any one of 6, 9, 12, 15, 18, 21, or 24 months. In some embodiments, the anti-dsDNA/chromatin antibody is stable at for about the same duration (e.g., ±about 2, 5, or 10%) as a native human antibody that specifically binds dsDNA or chromatin in given conditions (e.g., temperature, buffer).

In some embodiments, the anti-dsDNA/chromatin antibody is labeled, either directly or indirectly (e.g., with a secondary antibody or other indirect method such as Protein A, G, A/G, or strep-bio). In some embodiments, the anti-dsDNA/chromatin antibody is recognized by (specifically bound by) a labeled secondary antibody. In some embodiments, the secondary antibody is specific for human antibodies (anti-human). In some embodiments, both the anti-dsDNA/chromatin antibody and secondary antibody are labeled, e.g., with different labels. In some embodiments, the label is fluorescent.

In some embodiments, the anti-dsDNA/chromatin antibody specifically binds human dsDNA and human chromatin. In some embodiments, the chimeric anti-dsDNA/chromatin antibody specifically binds dsDNA in a non-sequence specific manner.

In some embodiments, the anti-dsDNA/chromatin antibody has a linear dilution profile within a range of 10-9000 relative fluorescence intensity (RFI), 100-9000 RFI, 1000-9000 RFI, 10-5000 RFI, 100-2500 RFI, or 50-5000 RFI for dsDNA. In some embodiments, the anti-dsDNA/chromatin antibody has a linear dilution profile within a range of 10-1500 RFI, 10-1000 RFI, 50-1000 RFI, 100-1500 RFI, 10-500 RFI, or 50-500 RFI for chromatin.

In some embodiments, the anti-dsDNA/chromatin antibody is in a solution with at least one additional antibody specific for a different target, e.g., a different nuclear antigen target. In some embodiments the solution comprises an anti-dsDNA/chromatin antibody as described herein and at least one additional antibody that specifically binds a target (antigen) from the cell nucleus or nucleolus, e.g., an antigen selected from the group consisting of: ribosomal protein, SS-A52, SS-A60, SS-B, Sm, Sm/ribonuclear protein (RNP), RNP-A, RNP-68, Scl-70, Jo-1, and centromere B. In some embodiments, the at least one additional antibody is derived from a human antibody, or has a constant region derived from a human antibody. In some embodiments, the solution includes at least 2, 3, 4, 5, 6, 7, 8, 9, 10, or 11 additional antibodies in any combination. In some embodiments, the anti-dsDNA/chromatin antibody and at least one additional antibody are recognized (specifically bound) by the same secondary antibody. In some embodiments, the anti-dsDNA/chromatin antibody and at least one additional antibody are recognized by different secondary antibodies (e.g., with different labels).

Further provided are kits for determining the amount of a sample (test) antibody that specifically binds dsDNA and/or chromatin, wherein the kit comprises at least one container with a defined (known) amount of anti-dsDNA/chromatin antibody. In some embodiments, the sample antibody is in or is obtained from a biological sample from a human. In some embodiments, the kit includes a container for the sample antibody and optionally a device for obtaining the biological sample. In some embodiments, the kit includes a labeled secondary antibody, e.g., an anti-human secondary antibody. In some embodiments, the anti-dsDNA/chromatin antibody is labeled, e.g., with a different label than the secondary antibody label. In some embodiments, the kit comprises two or more containers comprising the anti-dsDNA/chromatin antibody, wherein each of the two or more containers holds a different defined (known) amount of the anti-dsDNA/chromatin antibody. In some embodiments, the kit further comprises at least one container of dsDNA and/or chromatin, e.g., in a known amount.

In some embodiments, the kit also includes at least one container comprising a defined amount of at least one additional antibody that specifically binds a different target than the anti-dsDNA/chromatin antibody. In some embodiments, the target of the at least one additional antibody is selected from the group consisting of ribosomal protein, SS-A52, SS-A60, SS-B, Sm, Sm/ribonuclear protein (RNP), RNP-A, RNP-68, Scl-70, Jo-1, and centromere B. In some embodiments, the kit includes at least 2, 3, 4, 5, 6, 7, 8, 9, 10, or 11 additional antibodies in any combination. In some embodiments, the kit further comprises at least one container with the target of the at least one additional antibody.

Further provided are methods for generating a calibration curve (e.g., a standard or reference) for the anti-dsDNA/chromatin antibody. In some embodiments, the method comprises contacting an anti-dsDNA/chromatin antibody as described herein at a first known amount with dsDNA or chromatin in a first solution, detecting binding of the anti-dsDNA/chromatin antibody to the dsDNA or chromatin, and assigning a detection value to the first known amount; contacting the anti-dsDNA/chromatin antibody at a second known amount with dsDNA or chromatin in a second solution, wherein the dsDNA or chromatin is present at the same amount in the first and second solutions, detecting the binding of the anti-dsDNA/chromatin antibody to the dsDNA or chromatin, and assigning a second detection value to the second known amount of anti-dsDNA/chromatin antibody, thereby generating a calibration curve of the anti-dsDNA/chromatin antibody.

In some embodiments, the method further comprises repeating the steps of contacting, detecting, and assigning additional detection values for additional known amounts of anti-dsDNA/chromatin antibody, wherein the dsDNA or chromatin is present at the same amount in each of the solutions. In some embodiments, the steps of contacting, detecting, and assigning detection values are repeated for 3, 4, 5, 6, 7, 8, 9, or 10 known amounts of the anti-dsDNA/chromatin antibody. In some embodiments, the dsDNA or chromatin is attached to a substrate (e.g. a solid or semi-solid matrix, e.g., multiwell plate or bead). In some embodiments, the detecting comprises contacting the anti-dsDNA/chromatin antibody with a labeled secondary antibody.

In some embodiments, the method comprises contacting the anti-dsDNA/chromatin antibody with dsDNA, and the known amounts of anti-dsDNA/chromatin antibody are in the range of 0.001 to 100 ug/mL, e.g., 0.01 to 20 ug/mL, 0.01 to 5 ug/mL or about 0.05 to 0.5 ug/mL. In some embodiments, the method comprises contacting the anti-dsDNA/chromatin antibody with chromatin, and the known amounts of anti-dsDNA/chromatin antibody are in the range of 0.01 to 0.5 ug/mL.

Further provided is a calibration curve generated using the methods described above.

Provided herein is a method of detecting the presence of or determining the amount of a sample (e.g., test, unknown) antibody that specifically binds dsDNA or chromatin comprising: contacting an anti-dsDNA/chromatin antibody as described herein at a first known amount with dsDNA or chromatin in a first solution, detecting binding of the anti-dsDNA/chromatin antibody to the dsDNA or chromatin, and assigning a detection value to the first known amount; contacting the anti-dsDNA/chromatin antibody at a second known amount with dsDNA or chromatin in a second solution, wherein the dsDNA or chromatin is present at the same amount in the first and second solutions, detecting the binding of the anti-dsDNA/chromatin antibody to the dsDNA or chromatin, and assigning a second detection value to the second known amount of anti-dsDNA/chromatin antibody; contacting the sample antibody with dsDNA or chromatin in a test solution, detecting binding of the sample antibody to the dsDNA or chromatin, and assigning a detection value to the sample antibody; and comparing the detection value of the sample antibody to the first and second detection values, wherein the dsDNA or chromatin is present at the same amount in each of the solutions, thereby detecting the presence of or determining the amount of the sample antibody. In some embodiments, the binding of the sample antibody with dsDNA or chromatin is not detected, indicating that a sample antibody that specifically binds dsDNA or chromatin is not present.

In some embodiments, the method further comprises repeating the steps of contacting, detecting, and assigning additional detection values for additional known amounts of anti-dsDNA/chromatin antibody, and comparing the detection value of the sample antibody to the additional detection values, wherein the dsDNA or chromatin is present at the same amount in each of the solutions. In some embodiments, the steps of contacting, detecting, and assigning detection values are repeated for 3, 4, 5, 6, 7, 8, 9, or 10 known amounts of the anti-dsDNA/chromatin antibody. In some embodiments, the dsDNA or chromatin is attached to a substrate (e.g. a solid or semi-solid matrix, e.g., multiwell plate or bead). In some embodiments, the detecting comprises contacting the chimeric anti-dsDNA/chromatin antibody and/or sample antibody with a labeled secondary antibody. In some embodiments, the same secondary antibody is used to detect both the chimeric anti-dsDNA/chromatin antibody and the sample antibody.

In some embodiments, the method comprises contacting the anti-dsDNA/chromatin antibody and sample antibody with dsDNA, and the known amounts of anti-dsDNA/chromatin antibody are in the range of 0.01 to 10 ug/mL. In some embodiments, the method comprises contacting the anti-dsDNA/chromatin antibody and sample antibody with chromatin, and the known amounts of anti-dsDNA/chromatin antibody are in the range of 0.01 to 0.5 ug/mL.

In some embodiments, the sample antibody is obtained from or is in a biological sample from a human. In some embodiments, the method further comprises determining whether the human has an autoimmune disorder based on the amount or presence of the sample antibody. For example, the method can comprise diagnosing an autoimmune disorder in the human where the sample antibody is detected. In some embodiments, the autoimmune disease is selected from the group consisting of systemic lupus erythematosus, mixed connective tissue disease, sjogren's syndrome, scleroderma, dermatomyositis, polymyositis, and CREST syndrome, rheumatoid arthritis, juvenile arthritis, and Felty's syndrome.

### BRIEF DESCRIPTION OF THE DRAWINGS

FIG. 1 is a standard depiction of a tetrameric antibody structure with two light chains and two heavy chains. The variable region is shown as the top portion of each chain. The antibody on the right is chimeric, with a variable region derived from a mouse antibody and a constant region derived from a human antibody

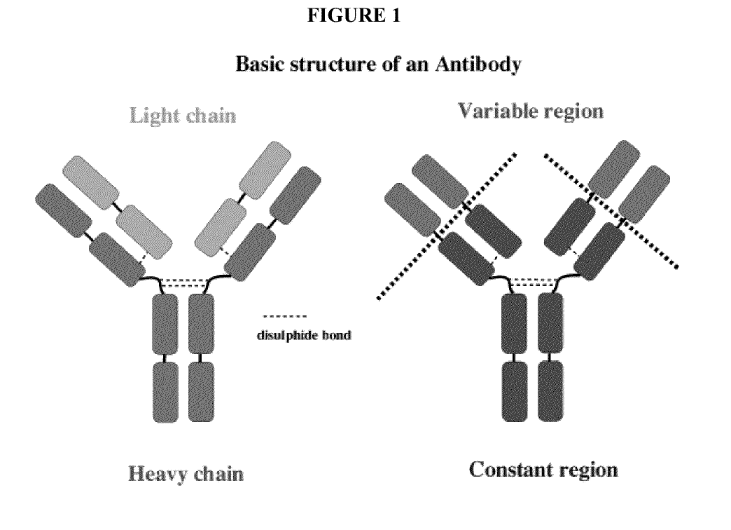

FIG. 2 shows stability of antibody binding to dsDNA at 25° C. over 14 days (equivalent to 5.6 months at 5° C.). (A) compares the signal retained versus day 0 signal for each antibody. (B) compares signal retained versus day-matched 5° C. signal for each antibody. Positive control antibodies, derived from native antibodies specific for dsRNA and chromatin, are designated Calib L04, L05, and L06. GG 1:50 refers to Glycine-HCL Glycerol eluted Chimeric antibody clone 20, at a 1:50 dilution. GG 1:100 refers to Glycine-HCL Glycerol eluted Chimeric antibody clone 20, at a 1:100 dilution. GT 1:25 refers to Glycyltyrosine eluted Chimeric antibody clone 20, at a 1:25 dilution.

FIG. 3 shows stability of antibody binding to dsDNA at 37° C. over 14 days (equivalent to 20.5 months at 5° C.). (A) compares the signal retained versus day 0 signal for each antibody. (B) compares signal retained versus day-matched 5° C. signal for each antibody. The antibodies are designated as in FIG. 2.

FIG. 4 shows stability of antibody binding to chromatin at 25° C. over 14 days (equivalent to 5.6 months at 5° C.). (A) compares the signal retained versus day 0 signal for each antibody. (B) compares signal retained versus day-matched 5° C. signal for each antibody. The antibodies are designated as in FIG. 2.

FIG. 5 shows stability of antibody binding to chromatin at 37° C. over 14 days (equivalent to 20.5 months at 5° C.). (A) compares the signal retained versus day 0 signal for each antibody. (B) compares signal retained versus day-matched 5° C. signal for each antibody. The antibodies are designated as in FIG. 2.

FIG. 6 shows linear signal to concentration relationship for chimeric antibody clones 20, 22, and 28. Plateau for clone 20 is due to detector limit. RFI: relative fluorescence intensity.

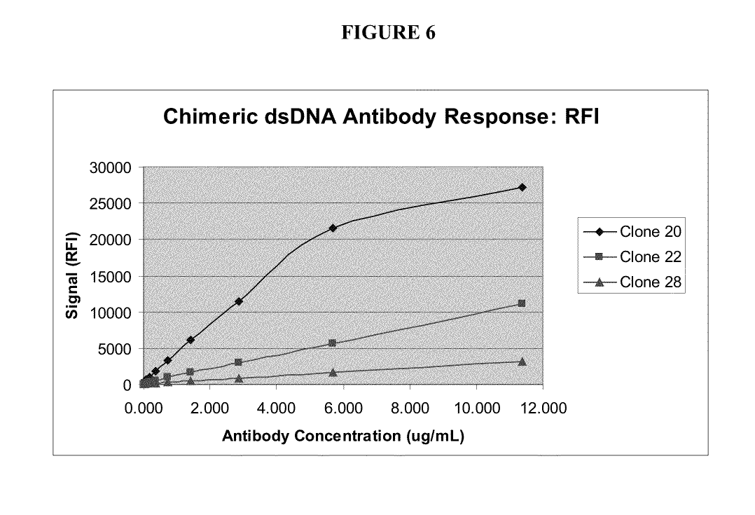

FIG. 7 shows linear signal (dsDNA) to concentration relationship for chimeric antibody clones 20, 22, and 28. RFI: relative fluorescence intensity.

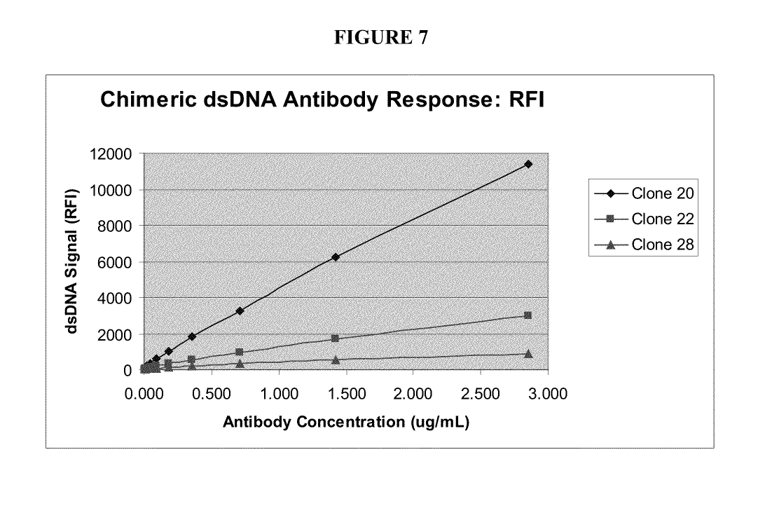

FIG. 8 shows linear signal (chomatin) to concentration relationship for chimeric antibody clones 20, 22, and 28. RFI: relative fluorescence intensity.

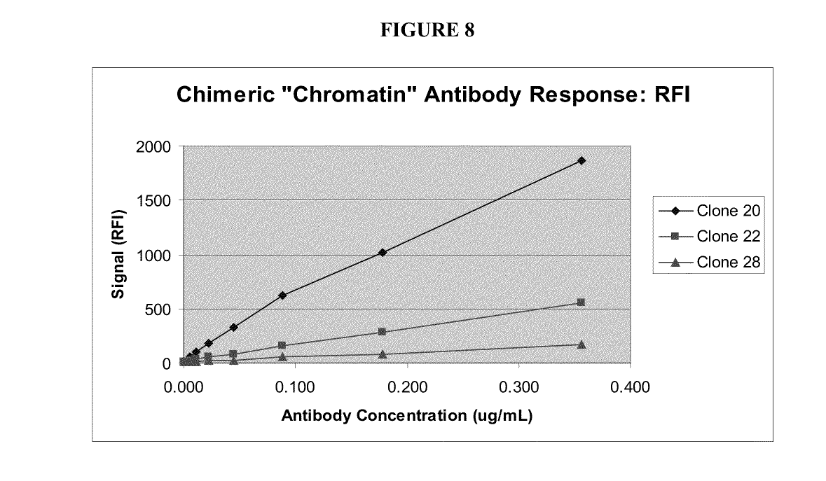

FIG. 9 shows linear signal from both dsDNA and chromatin with chimeric antibody clone 20. Note that the typical assay range for dsDNA is 10-9000 RFI (0-300 IU/mL), while it is 10-1500 RFI (0-8 AI (antibody index)) for chromatin.

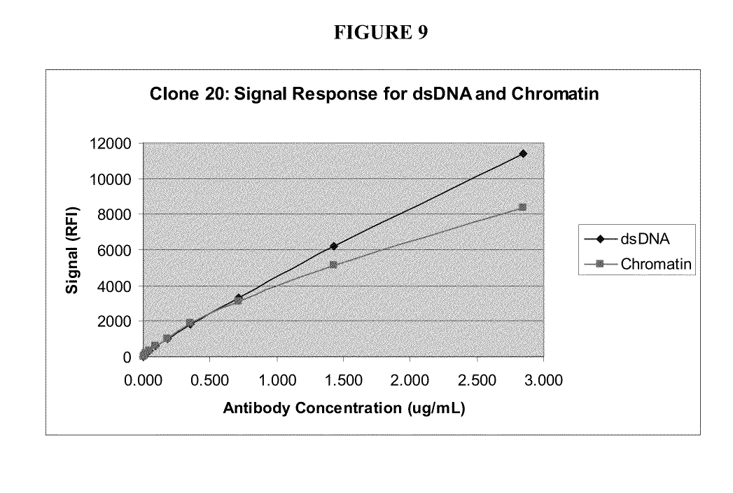

### DETAILED DESCRIPTION OF THE INVENTION

### I. Introduction

Provided herein are chimeric monoclonal antibodies that specifically bind to dsDNA and chromatin. Naturally-occurring antibodies specific for dsDNA and/or chromatin are typically not found in high concentrations, and have variable binding characteristics (e.g., affinity, avidity, epitope, etc.). The presently described antibodies can be clonally or recombinantly expressed, thus providing a reliable source of antibodies with known binding characteristics. The presently described antibodies are stable in storage and assay conditions, and can detect dsDNA and chromatin in the same linear concentration range. The presently described chimeric antibodies can be designed to have varying affinity for dsDNA and chromatin, e.g., one chimeric anti-dsDNA/chromatin antibody can have a higher affinity for dsDNA relative to chromatin, while another has a higher affinity for chromatin relative to dsDNA. The presently described chimeric antibodies have similar stability and linear binding curves as native autoantibodies that are commonly used for calibration. Because the presently described antibodies are chimeric (e.g., with a constant region from a human antibody), the same secondary antibody can be used to detect or separate native antibodies (e.g., from a human) and the presently described antibodies.

### II. Definitions

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4^{th} ed. 2007); Sambrook et al., MOLECULAR CLONING, A LABORATORY MANUAL, Cold Springs Harbor Press (Cold Springs Harbor, N.Y. 1989). The term “a” or “an” is intended to mean “one or more.” The term “comprise” and variations thereof such as “comprises” and “comprising,” when preceding the recitation of a step or an element, are intended to mean that the addition of further steps or elements is optional and not excluded. Any methods, devices and materials similar or equivalent to those described herein can be used in the practice of this invention. The following definitions are provided to facilitate understanding of certain terms used frequently herein and are not meant to limit the scope of the present disclosure.

The term “autoantibody” refers to an antibody produced by an individual that specifically binds an epitope in the same individual. Autoantibodies can be described as directed against “self” antigens, and can be indicative of an autoimmune disease. For example, individuals with multiple sclerosis produce autoantibodies that specifically bind a component of the myelin sheath that normally protects nerve cells. Autoantibody binding in MS patients results in recruitment of immune cells that damage and degrade the myelin, and subsequent damage to the underlying nerve cells.

The term Anti-Nuclear Antibody (ANA) refers to an antibody that specifically binds a substance normally found in a cell nucleus, e.g., dsDNA, chromatin, ribosomal proteins, centromeric proteins (e.g., Centromere B), SS-A, SS-B, Sm, Sm/RNP, RNP, Scl-70, Jo-1, etc. The presence of ANAs that are also autoantibodies in an individual can be indicative of particular autoimmune conditions, e.g., systemic lupus erythematosus (SLE), mixed connective tissue disease (MCTD), Sjogren's syndrome (SS), scleroderma (systemic sclerosis), dermatomyositis (DM), polymyositis (PM), CREST syndrome, rheumatoid arthritis, juvenile arthritis, Felty's syndrome, etc.

The term “nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA.

The term “double stranded DNA” or “dsDNA” is intended to refer to a deoxyribonucleotide polymer (DNA strand) hybridized to its complement through Watson-Crick bonding. The dsDNA can be of any length and can be associated with additional components (e.g., histone proteins or proteins involved in replication or transcription). One of skill will appreciate that the two strands of DNA may not be 100% complementary, so long as the percentage is high enough in the given conditions for the two strands to remain associated.

Chromatin is a combination of dsDNA and proteins that condenses to form chromosomes. Chromatin can be “unpacked” so that the DNA is accessible, e.g., while a gene on the DNA is expressed and transcribed. Chromatin proteins include histones, which can be modified, e.g., methylated or acetylated.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

A variety of methods of specific DNA and RNA measurements that use nucleic acid hybridization techniques are known to those of skill in the art (see, Sambrook, Id.). Some methods involve electrophoretic separation (e.g., Southern blot for detecting DNA, and Northern blot for detecting RNA), but measurement of DNA and RNA can also be carried out in the absence of electrophoretic separation (e.g., quantitative PCR, dot blot, or array).

The words “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an α carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following amino acids are typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The terms “identical” or “percent identity,” in the context of two or more nucleic acids, or two or more polypeptides, refer to two or more sequences or subsequences that are the same or have a specified percentage of nucleotides, or amino acids, that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters, or by manual alignment and visual inspection. See e.g., the NCBI web site at ncbi.nlm.nih.gov/BLAST. Such sequences are then said to be “substantially identical.” Percent identity is typically determined over optimally aligned sequences, so that the definition applies to sequences that have deletions and/or additions, as well as those that have substitutions. The algorithms commonly used in the art account for gaps and the like. Typically, identity exists over a region comprising an antibody epitope, or a sequence that is at least about 25 amino acids or nucleotides in length, or over a region that is 50-100 amino acids or nucleotides in length, or over the entire length of the reference sequence.

The term “recombinant” when used with reference, e.g., to a cell, or nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous,” with reference to a polynucleotide or polypeptide, indicates that the polynucleotide or polypeptide comprises two or more subsequences that are not found in the same relationship to each other in nature. For instance, a heterologous polynucleotide or polypeptide is typically recombinantly produced, having two or more sequences from unrelated genes arranged to make a new functional unit, e.g., a promoter from one source and a coding region from another source. Similarly, a heterologous protein indicates that the protein comprises two or more subsequences that are not found in the same relationship to each other in nature (e.g., a fusion protein).

The term “native” or “naturally occurring” refers to a substance (e.g., protein, antibody, nucleic acid) that is not modified from its natural form. A native or naturally occurring substance can, however, be isolated from its natural environment.

The term “primary antibody” will be understood by one of skill to refer to an antibody or fragment thereof that specifically binds to an analyte (e.g., substance, antigen, component) of interest. The primary antibody can further comprise a tag, e.g., for recognition by a secondary antibody or associated binding protein (e.g., GFP, biotin, or strepavidin), or to facilitate separation (e.g., a poly-His tag).

The term “secondary antibody” refers to an antibody that specifically binds to a primary antibody. A secondary antibody can be specific for the primary antibody (e.g., specific for primary antibodies derived from a particular species) or a tag on the primary antibody (e.g., GFP, biotin, or strepavidin). Secondary antibodies are usually attached to a detectable moiety or a matrix for separation (e.g., a bead, chromatography agent, array, or ELISA plate).

The term “derived from,” with reference to an antibody, indicates that the antibody was originally isolated from cells of that type. For example, an antibody derived from a mouse is one that was originally obtained from a mouse, or mouse cell, but may have been further manipulated (e.g., labeled, recombinantly expressed, humanized, etc.). One of skill will understand that, in the case of a full length tetramer antibody, the Fc region of the antibody can have species-specific sequences that can be targeted for specific recognition, e.g., by a secondary antibody.

The term “antibody” refers to a polypeptide structure, e.g., an immunoglobulin, conjugate, or fragment thereof that retains antigen binding activity. The term includes but is not limited to polyclonal or monoclonal antibodies of the isotype classes IgA, IgD, IgE, IgG, and IgM, derived from human or other mammalian cells, including natural or genetically modified forms such as humanized, human, single-chain, chimeric, synthetic, recombinant, hybrid, mutated, grafted, and in vitro generated antibodies. The term encompasses conjugates, including but not limited to fusion proteins containing an immunoglobulin moiety (e.g., chimeric or bispecific antibodies or scFv's), and fragments, such as Fab, F(ab′)2, Fv, scFv, Fd, dAb and other compositions.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively. The variable region contains the antigen-binding region of the antibody (or its functional equivalent) and is most critical in specificity and affinity of binding. See Paul, Fundamental Immunology (2003).

Antibodies can exist as intact immunoglobulins or as any of a number of well-characterized fragments that include specific antigen-binding activity. Such fragments can be produced by digestion with various peptidases. Pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies or those identified using phage display libraries (see, e.g., McCafferty et al., Nature 348:552-554 (1990)).

As used herein, the term “Fv” refers to a monovalent or bi-valent variable region fragment, and can encompass only the variable regions (e.g., V_{L} and/or V_{H}), as well as longer fragments, e.g., an Fab, Fab′ or F(ab′)2, which also includes C_{L} and/or C_{H}1. Unless otherwise specified, the term “Fc” refers to a heavy chain monomer or dimer comprising C_{H}1 and C_{H}2 regions.

A single chain Fv (scFv) refers to a polypeptide comprising a V_{L} and V_{H} joined by a linker, e.g., a peptide linker. ScFvs can also be used to form tandem (or di-valent) scFvs or diabodies. Production and properties of tandem scFvs and diabodies are described, e.g., in Asano et al. (2011) J Biol. Chem. 286:1812; Kenanova et al. (2010) Prot Eng Design Sel 23:789; Asano et al. (2008) Prot Eng Design Sel 21:597.

A “monoclonal antibody” refers to a clonal preparation of antibodies with a single binding specificity and affinity for a given epitope on an antigen. A “polyclonal antibody” refers to a preparation of antibodies that are raised against a single antigen, but that includes antibodies with different binding specificities and affinities for epitopes on the single antigen.

As used herein, “V-region” refers to an antibody variable region domain comprising the segments of Framework 1, CDR1, Framework 2, CDR2, and Framework 3, including CDR3 and Framework 4, which segments are added to the V-segment as a consequence of rearrangement of the heavy chain and light chain V-region genes during B-cell differentiation.

As used herein, “complementarity-determining region (CDR)” refers to the three hypervariable regions in each chain that interrupt the four “framework” regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

The sequences of the framework regions of different light or heavy chains are relatively conserved within a species. The framework region of an antibody, that is the combined framework regions of the constituent light and heavy chains, serves to position and align the CDRs in three dimensional space.

The amino acid sequences of the CDRs and framework regions can be determined using various well known definitions in the art, e.g., Kabat, Chothia, international ImMunoGeneTics database (IMGT), and AbM (see, e.g., Johnson et al., supra; Chothia & Lesk, (1987) J. Mol. Biol. 196, 901-917; Chothia et al. (1989) Nature 342, 877-883; Chothia et al. (1992) J. Mol. Biol. 227, 799-817; Al-Lazikani et al., J. Mol. Biol 1997, 273(4)). A helpful guide for locating CDRs using the Kabat system can be found at the website available at bioinf.org.uk/abs. Definitions of antigen combining sites are also described in the following: Ruiz et al. Nucleic Acids Res., 28, 219-221 (2000); and Lefranc Nucleic Acids Res. January 1; 29(1):207-9 (2001); MacCallum et al., J. Mol. Biol., 262: 732-745 (1996); and Martin et al, Proc. Natl Acad. Sci. USA, 86, 9268-9272 (1989); Martin, et al, Methods Enzymol., 203: 121-153, (1991); Pedersen et al, Immunomethods, 1, 126, (1992); and Rees et al, In Sternberg M. J. E. (ed.), Protein Structure Prediction. Oxford University Press, Oxford, 141-172 1996).

A “chimeric antibody” refers to an antibody in which (a) the constant region, or a portion thereof, is altered, replaced or exchanged so that the antigen binding site (variable region, CDR, or portion thereof) is linked to a constant region of a different or altered class, effector function and/or species; or (b) the variable region, or a portion thereof, is altered, replaced or exchanged with a variable region having a different or altered antigen specificity (e.g., CDR and framework regions from different species). Chimeric antibodies can include variable region fragments, e.g., a recombinant antibody comprising two Fab or Fv regions or an scFv. A chimeric antibody can also, as indicated above, include an Fc region from a different source than the attached Fv regions. In some cases, the chimeric antibody includes chimerism within the Fv region. An example of such a chimeric antibody would be a humanized antibody where the FRs and CDRs are from different sources.

The terms “antigen,” “immunogen,” “antibody target,” “target analyte,” and like terms are used herein to refer to a molecule, compound, or complex that is recognized by an antibody, i.e., can be specifically bound by the antibody. The term can refer to any molecule that can be specifically recognized by an antibody, e.g., a polynucleotide, polypeptide, carbohydrate, lipid, chemical moiety, or combinations thereof (e.g., phosphorylated or glycosylated polypeptides, etc.). One of skill will understand that the term does not indicate that the molecule is immunogenic in every context, but simply indicates that it can be targeted by an antibody.

Antibodies bind to an “epitope” on an antigen. The epitope is the localized site on the antigen that is recognized and bound by the antibody. Protein epitopes can include a few amino acids or portions of a few amino acids, e.g., 5 or 6, or more, e.g., 20 or more amino acids, or portions of those amino acids. Epitopes can also include non-protein components, e.g., nucleic acid (e.g., RNA or DNA), carbohydrate, or lipid. Epitopes can also include combinations of these components. In some cases, the epitope is a three-dimensional moiety. Thus, for example, where the target is a protein target, the epitope can be comprised of consecutive amino acids, or amino acids from different parts of the protein that are brought into proximity by protein folding (e.g., a discontinuous epitope). The same is true for other types of target molecules, such as dsDNA and chromatin, that form three-dimensional structures.

The terms “specific for,” “specifically binds,” and like terms refer to a molecule (e.g., antibody or antibody fragment) that binds to a target with at least 2-fold greater affinity than non-target compounds, e.g., at least 4-fold, 5-fold, 6-fold, 7-fold, 8-fold, 9-fold, 10-fold, 20-fold, 25-fold, 50-fold, or 100-fold greater affinity. For example, an antibody that specifically binds a particular target will typically bind the target with at least a 2-fold greater affinity than a non-target.

The term “binds” with respect to an antibody target (e.g., antigen, analyte, epitope), typically indicates that an antibody binds a majority of the antibody targets in a pure population, assuming an appropriate molar ratio of antibody to target. For example, an antibody that binds a given antibody target typically binds to at least ⅔ of the antibody targets in a solution (e.g., 75, 80, 85, 90, 91, 92, 93, 94, 95, 96, 97, 98, 99, or 100%). One of skill will recognize that some variability will arise depending on the method and/or threshold of determining binding.

As used herein, a first antibody, or an antigen-binding portion thereof, “competes” for binding to a target with a second antibody, or an antigen-binding portion thereof, when binding of the second antibody with the target is detectably decreased in the presence of the first antibody compared to the binding of the second antibody in the absence of the first antibody. The reverse, where the binding of the first antibody to the target is also detectably decreased in the presence of the second antibody, can exist, but need not be the case. That is, a second antibody can inhibit the binding of a first antibody to the target without that first antibody inhibiting the binding of the second antibody to the target. However, where each antibody detectably inhibits the binding of the other antibody to its cognate epitope or ligand, whether to the same, greater, or lesser extent, the antibodies are said to “cross-compete” with each other for binding of their respective epitope(s). Both competing and cross-competing antibodies are encompassed by the present invention. The term “competitor” antibody can be applied to the first or second antibody as can be determined by one of skill in the art. In some cases, the presence of the competitor antibody (e.g., the first antibody) reduces binding of the second antibody to the target by at least 10%, e.g., 20%, 30%, 40%, 50%, 60%, 70%, 80%, or more, e.g., so that binding of the second antibody to target is undetectable in the presence of the first (competitor) antibody.

The terms “label,” “detectable moiety,” “detectable agent,” and like terms refer to a composition detectable by spectroscopic, photochemical, biochemical, immunochemical, chemical, or other physical means. For example, useful labels include fluorescent dyes, luminescent agents, radioisotopes (e.g., ³²P, ³H), electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target analyte. Any method known in the art for conjugating an antibody to the label may be employed, e.g., using methods described in Hermanson, Bioconjugate Techniques 1996, Academic Press, Inc., San Diego. The term “tag” can be used synonymously with the term “label,” but generally refers to an affinity-based moiety, e.g., a “His tag” for purification, or a “strepavidin tag” that interacts with biotin.

A “labeled” molecule (e.g., nucleic acid, protein, or antibody) is one that is bound, either covalently, through a linker or a chemical bond, or noncovalently, through ionic, van der Waals, electrostatic, or hydrogen bonds to a label such that the presence of the molecule may be detected by detecting the presence of the label bound to the molecule.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare signal strength in given conditions, e.g., in the presence of a test antibody, in the absence of the test antibody (negative control), or in the presence of a known antibody with a known affinity (positive control). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The term “stable,” with reference to an antibody, indicates that the antibody retains a certain level of activity at given conditions (e.g., temperature, duration, pH, etc.). Activity can be expressed in terms of target binding (e.g., in terms of amount of target bound). Thus, an antibody can be considered stable if it retains at least any of 50%, 60%, 70%, 75%, 80%, 85%, 90%, 95% or higher target binding compared to a control. One of skill will appreciate that antibody activity, and stability, can be expressed using other criteria, e.g., structural criteria, target binding affinity, etc. The stability of the antibody can be considered with relation to time, so that antibody activity at a starting time is compared to activity at later times. The stability can also be considered in different buffer conditions, different states (e.g., pre- and post-lyophilization, pre- and post-freezing) or at different temperatures (e.g., activity at a control temperature compared to higher or lower temperatures).

The “amount” of a substance (e.g., antibody, target molecule, protein, nucleic acid, etc.) can be expressed as a relative term, e.g., compared to a defined or known amount, or as a percentage of a control or starting amount. For example, the amount of chromatin can be expressed according to Antibody Index (AI), an arbitrary comparative measure. The amount of dsDNA can be expressed using International Units (IU). Amounts can be expressed in terms of relative fluorescent intensity (RFI), in terms of concentration (e.g., mg/ml or molarity), according to mass or binding units, etc.

A calibration curve is a tool for determining the amount or concentration of a substance in a sample by comparing the unknown amount as detected to a set of standards of known amounts. Calibration curves reveal the limit of detection (LOD) and limit of linearity (LOL) for a given assay. In the context of the present disclosure, the substance of unknown amount can be an autoantibody from a patient sample, and the calibration standards are known amounts of an antibody specific for the same antigen. A “linear dilution profile,” as used herein, indicates that antibody activity (e.g., target binding) correlates with its concentration in a linear manner. One of skill will recognize that, within a range of detection, the calibration “curve” can be linear.

### III. Detection of Antibody Binding and Affinity

The chimeric anti-dsDNA/chromatin antibodies described herein can be used with any antibody-based assay or separation procedure, and are conveniently used as a standard for determining the amount of or binding ability of a test antibody. Thus, the known target and binding ability of the presently described antibodies can be used as a baseline comparison.

A. Detection of Antibody Binding

Antibody binding to a target can be detected using immunoassays, for example, enzyme linked immunoabsorbent assay (ELISA), fluorescent immunosorbent assay (FIA), immunohistochemistry, chemical linked immunosorbent assay (CLIA), radioimmuno assay (RIA), flow cytometry (e.g., fluorescence activated cell sorting or FACS), Western blot, and immunoblotting. Additional applicable immunotechniques include competitive and non-competitive assay systems, e.g., “sandwich” immunoassays, immunoprecipitation assays, precipitin reactions, immunodiffusion assays, immunoradiometric assays, fluorescent immunoassays, etc. For a review of applicable immunoassays, see, e.g., The Immunoassay Handbook, David Wild, ed., Stockton Press, New York, 1994; Ausubel et al, eds, 1994, Current Protocols in Molecular Biology, Vol. 1, John Wiley & Sons, Inc., New York.

Western blotting is usually used to detect the presence or relative amount of a given target. The technique generally comprises preparing protein samples, electrophoresis of the protein samples in a polyacrylamide gel (e.g., 8%-20% SDS-PAGE), transferring the proteins from the polyacrylamide gel to a membrane such as nitrocellulose, PVDF or nylon, blocking the membrane in blocking solution (e.g., PBS with 3% BSA or non-fat milk), washing the membrane, contacting the membrane with primary antibody diluted in blocking buffer, washing the membrane in washing buffer, incubating the membrane with a labeled secondary antibody diluted in blocking buffer, washing the membrane in wash buffer, and detecting the presence or amount of the target by detecting the presence or amount of the label.

ELISAs include a number of variations. In some cases, the ELISA comprises preparing a target antigen, coating the wells of a multiwell microtiter plate or other matrix material with the antigen, adding primary antibody, and incubating for a period of time, followed by addition of labeled secondary antibody. One of skill in the art would be knowledgeable as to other variations of ELISAs, e.g., where target is labeled and the primary antibody is coated on the matrix material, etc.

Immunoprecipitation and immunoseparation protocols can comprise contacting a sample with primary antibody specific for the desired target in the sample, incubating for a period of time (e.g., 1-4 hours at 4° C.), adding secondary antibody-coated sepharose beads (or other support matrix) to the mixture and incubating again, washing the beads, and resuspending the beads in an SDS/sample buffer or elution buffer. Again, one of skill will be familiar with variations of immunoprecipitations, e.g., using Protein A, Protein G, Protein A/G, secondary antibody, or target as the binding partner for primary antibody.

Bead-based assays include a number of variations. For example, the BioPlex™ 2200 system can be employed with a target antigen bound to a fluoromagnetic bead with a distinct fluorescent signature. An aliquot of a patient sample (e.g. serum, plasma) is reacted with the bead. Patient antibodies binding specifically to the target antigen are detected by a fluorophore-labeled secondary antibody. Multiple bead classes (e.g. different fluorescent signatures), with different target antigens, can be used simultaneously or multiplexed. One of skill in the art would be knowledgeable as to other variations of bead-based assays, e.g., where an antibody may be bound to the bead for detecting an antigen in the patient sample. A fluorophore-labeled secondary antibody recognizing the antigen-antibody complex would act as a detector in this sandwich-assay format.

B. Labels

The chimeric anti-dsDNA/chromatin antibodies described herein can be conjugated or otherwise associated with a detectable label. In some embodiments, the chimeric anti-dsDNA/chromatin antibody (primary antibody) is detected using a secondary antibody that is conjugated or associated with a detectable label. The association can be direct e.g., a covalent bond, or indirect, e.g., using a secondary binding agent, chelator, or linker. The terms “detectable agent,” “detectable label,” “detectable moiety,” “label,” “imaging agent,” and like terms are used synonymously herein. In some embodiments, both the primary and secondary antibodies are labeled, e.g., with the same or with different labels.

In some embodiments, the label can include an optical agent such as a fluorescent agent, phosphorescent agent, chemiluminescent agent, etc. Numerous agents (e.g., dyes, probes, labels, or indicators) are known in the art and can be used in the present invention. (See, e.g., Invitrogen, The Handbook—A Guide to Fluorescent Probes and Labeling Technologies, Tenth Edition (2005)). Fluorescent agents can include a variety of organic and/or inorganic small molecules or a variety of fluorescent proteins and derivatives thereof. For example, fluorescent agents can include but are not limited to cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenopyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, and BODIPY™ derivatives.

The presently disclosed antibodies can be used for immunoassays, e.g., Western blots, ELISAs, FACS, immunoprecipitation, immunohistochemistry, immunofluorescence (e.g., using cells or tissue from a cell line or patient sample). In some embodiments, cells or cellular material used in the immunoassay is fixed. In some embodiments, cells or cellular material is not fixed.

A radioisotope can be used as a label, and can include radionuclides that emit gamma rays, positrons, beta and alpha particles, and X-rays. Suitable radionuclides include but are not limited to ²²⁵Ac, ⁷²As, ²¹¹At, ¹¹B, ¹²⁸Ba, ²¹²Bi, ⁷⁵Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga ³H, ¹⁶⁶Ho, ¹²³I, ¹²⁴I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ^{99m}Tc, ⁸⁸Y and ⁹⁰Y. In certain embodiments, radioactive agents can include ¹¹¹In-DTPA, ^{99m}Tc(CO)₃-DTPA, ^{99m}Tc(CO)₃-ENPy2, ^{62/64/67}Cu-TETA, ^{99m}Tc(CO)₃-IDA, and ^{99m}Tc(CO)₃triamines (cyclic or linear). In other embodiments, the agents can include DOTA and its various analogs with ¹¹¹In, ¹⁷⁷Lu, ¹⁵³Sm, ^{88/90}Y, ^{62/64/67}Cu, or ^{67/68}Ga. I

In some embodiments, the antibody can be associated with a secondary binding ligand or to an enzyme (an enzyme tag) that will generate a colored product upon contact with a chromogenic substrate. Examples of suitable enzymes include urease, alkaline phosphatase, (horseradish) hydrogen peroxidase (HRP) and glucose oxidase. Secondary binding ligands include, e.g., biotin and avidin or streptavidin, as known in the art. In some embodiments, the label is a fluorescent protein sequence, and can be recombinantly combined with the antibody polypeptide sequence.

Techniques for conjugating detectable agents to antibodies are well known and antibody labeling kits are commercially available from dozens of sources (e.g., Invitrogen, Pierce, Sigma Aldrich, Biotium, Jackson Immunoresearch, etc.). A review of common protein labeling techniques can be found in Biochemical Techniques: Theory and Practice (1987).

Antibodies are generally labeled in an area that does not interfere with target binding, or in this case, with stability of the immune complex. In some embodiments, the detectable moiety is attached to the constant region, or outside the CDRs in the variable region. One of skill in the art will recognize that the optimal position for attachment may be located elsewhere on the antibody, so the position of the detectable moiety can be adjusted accordingly. In some embodiments, the ability of the antibody to associate with the epitope is compared before and after attachment to the detectable moiety to ensure that the attachment does not unduly disrupt binding.

C. Affinity

The presently described chimeric anti-dsDNA/chromatin antibodies typically bind to the target (dsDNA or chromatin) with a binding affinity of about 10⁶, 10⁷, 10⁸, 10⁹, 10¹⁰, 10¹¹, or 10¹² M⁻¹ (e.g., with a Kd in the micromolar (10⁻⁶), nanomolar (10⁻⁹), picomolar (10⁻¹²), or lower range). In some embodiments, the affinity of the chimeric anti-dsDNA/chromatin antibody for its target will be similar to native antibodies generated against the same target (e.g., autoantibodies generated against dsDNA or chromatin). In some embodiments, the affinities will be similar, e.g., within one order of magnitude. In some embodiments, the affinity is expressed in terms of Kd, wherein

Kd=[antibody]×[target]/[antibody-target complex].

For example, the “antibody” in the above equation can refer to a chimeric antibody as described herein, the “target” can refer to dsDNA or chromatin, and the antibody-target complex can refer to a complex comprising the chimeric antibody bound to dsDNA or chromatin. One of skill will understand that a higher affinity will correspond to a lower Kd (reduced dissociation).

The specificity of antibody binding can be defined in terms of the comparative dissociation constants (Kd) of the antibody for the target as compared to the dissociation constant with respect to the antibody and other materials in the environment or unrelated molecules in general. Typically, the Kd for the antibody with respect to the unrelated material will be at least 2-fold, 3-fold, 4-fold, 5-fold, 10-fold, 20-fold, 50-fold, 100-fold, 200-fold or higher than Kd with respect to the target.

A targeting moiety will typically bind with a Kd of less than about 1000 nM, e.g., less than 250, 100, 50, 20 or lower nM. In some embodiments, the Kd of the affinity agent is less than 15, 10, 5, or 1 nM. In some embodiments, the Kd is 1-100 nM, 0.1-50 nM, 0.1-10 nM, or 1-20 nM. The value of the dissociation constant (Kd) can be determined by well-known methods, and can be computed even for complex mixtures by methods as disclosed, e.g., in Caceci et al., Byte (1984) 9:340-362.

Affinity of an antibody, or any targeting agent, for a target can be determined according to methods known in the art, e.g., as reviewed in Ernst et al. Determination of Equilibrium Dissociation Constants, Therapeutic Monoclonal Antibodies (Wiley & Sons ed. 2009).

Quantitative ELISA, and similar array-based affinity methods can be used. ELISA (Enzyme linked immunosorbent signaling assay) is an antibody-based method. In some cases, an antibody specific for target of interest is affixed to a substrate, and contacted with a sample suspected of containing the target. The surface is then washed to remove unbound substances. Target binding can be detected in a variety of ways, e.g., using a second step with a labeled antibody, direct labeling of the target, or labeling of the primary antibody with a label that is detectable upon antigen binding. In some cases, the antigen is affixed to the substrate (e.g., using a substrate with high affinity for proteins, or a Strepavidin-biotin interaction) and detected using a labeled antibody (or other targeting moiety). Several permutations of the original ELISA methods have been developed and are known in the art (see Lequin (2005) Clin. Chem. 51:2415-18 for a review).

The Kd, Kon, and Koff can also be determined using surface plasmon resonance (SPR). SPR techniques are reviewed, e.g., in Hahnfeld et al. Determination of Kinetic Data Using SPR Biosensors, Molecular Diagnosis of Infectious Diseases (2004). In a typical SPR experiment, one interactant (target or targeting agent) is immobilized on an SPR-active, gold-coated glass slide in a flow cell, and a sample containing the other interactant is introduced to flow across the surface. When light of a given frequency is shined on the surface, the changes to the optical reflectivity of the gold indicate binding, and the kinetics of binding.

Binding affinity can also be determined by anchoring a biotinylated interactant to a streptaviden (SA) sensor chip. The other interactant is then contacted with the chip and detected, e.g., as described in Abdessamad et al. (2002) Nuc. Acids Res. 30:e45.

Binding affinity can also be determined using comparative methods. For example, a set of components with known affinities can be compared to the test components (i.e., antibody and target) under various conditions, e.g., wash conditions of various stringencies.

### IV. Production of Chimeric Antibodies

Many techniques known in the art can be used for production of antibodies as described herein (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)).

Methods for production and modification of chimeric anti-dsDNA/chromatin antibodies as described herein are known in the art. For example, Beidler et al. (1988) J. Immunol. 141:4053 describes high level recombinant expression of a chimeric antibody with mouse variable regions and human constant regions. The recombinant antibody sequences were electroporated into a hybridoma cell line adapted to grow and produce antibody in serum free conditions.

Hand et al. (1994) Cancer 73:1106 review methods for generating chimeric antibodies, sFv antibodies, antibodies with altered subclass or glycosylation, and compare the properties of these antibody forms. Ono et al. (2003) describe production of a chicken scFv-human Fc (IgG1) fusion. The chimeric single chain antibody was highly expressed from a retroviral vector in CHO cells.

Oppezzo et al. (2000) Hybridoma 19:229 describe production of a mouse-human chimeric antibody, using human mu, gamma1, and kappa constant regions. The antibodies were expressed from a transfected cell line and separated using gel filtration chromatography. Knappick et al. (2009) Ann NY Acad Sci 1173:190 describe isolation of a human monoclonal antibody from a library, and the subsequent cloning and high level recombinant expression of the antibody using HuCAL.

The present antibodies can be produced using any number of expression systems, including prokaryotic and eukaryotic expression systems. In some embodiments, the expression system is a mammalian cell expression, such as a hybridoma, or a CHO cell expression system. Many such systems are widely available from commercial suppliers. In embodiments in which an antibody comprises both heavy and light chains, the heavy and light chains can be expressed using a single vector, e.g., in a di-cistronic expression unit, or under the control of different promoters. In other embodiments, the heavy and light chains can be expressed using separate vectors, or can be expressed in different cells and later combined.

### V. Autoimmune Diseases Characterized by Anti-dsDNA and Anti-Chromatin Antibodies

The presently described chimeric anti-dsDNA/chromatin antibodies can be used as part of a diagnostic assay, as a comparison for antibodies in a patient sample. If the sample includes antibodies that bind dsDNA or chromatin, the binding can be detected and compared to the binding of a known amount of the presently described antibodies.

The presence of such autoantibodies in a patient sample is indicative of certain autoimmune conditions including systemic lupus erythematosus (SLE), mixed connective tissue disease (MCTD), Sjogren's syndrome (SS), scleroderma (systemic sclerosis), dermatomyositis (DM), polymyositis (PM), CREST syndrome. Autoantibodies specific for dsDNA and/or chromatin are also found in rheumatoid arthritis, Felty's syndrome, and juvenile arthritis. A review of anti-dsDNA and anti-chromatin related disorders include Kavanaugh et al. (2002) Arthritis & Rheumatism 47:546.

### VI. Kits

The presently described chimeric antibodies specific for dsDNA and chromatin (chimeric anti-dsDNA/chromatin antibodies) can be included in a kit. In such embodiments, the chimeric anti-dsDNA/chromatin antibody is provided in a known amount and packaged, e.g., for shipping and storage (e.g., lyophilized, or in a buffer). The kit can be designed for calibrating the binding or affinity of test antibodies specific for dsDNA and/or chromatin, e.g., native antibodies of known specificity or antibodies from a sample that may or may not include antibodies specific for dsDNA and/or chromatin. In such cases, the kit will include appropriate instructions to prepare a calibration curve using a chimeric anti-dsDNA/chromatin antibody as described herein, or include multiple containers of the chimeric anti-dsDNA/chromatin antibody at appropriate dilutions to prepare a calibration curve. In some embodiments, the antibody included in the kit is labeled (directly or indirectly). In some embodiments, the kit includes a secondary antibody (e.g., detectably labeled) specific for the chimeric anti-dsDNA/chromatin antibody. In some embodiments, the secondary antibody is specific for both the chimeric anti-dsDNA/chromatin antibody and the intended test antibody. In some embodiments, more than one secondary antibody is included with the kit.

In some embodiments, the kit includes at least one tube or other container with a known amount of dsDNA. In some embodiments, the kit includes at least one tube or other container with a known amount of chromatin. In some embodiments, the dsDNA and/or chromatin is detectably labeled or attached to a matrix.

In some embodiments, the kit includes a chimeric anti-dsDNA/chromatin antibody as described herein and additional antibodies specific for different antigens. In some embodiments, the kit is designed for calibrating multiple antibodies with different targets. For example, the kit can include a chimeric anti-dsDNA/chromatin antibody as described herein, and at least one additional antibody specific for a different target, e.g., an autoimmune target such as other nuclear components. In some embodiments, the kit includes a known amount of the at least one additional antibody, packaged as described above for the anti-dsDNA/chromatin antibody. In some embodiments, the at least one additional antibody targets a nuclear or nucleolar antigen, e.g., an antigen selected from the group consisting of ribosomal protein, SS-A52, SS-A60, SS-B, Sm, Sm/RNP, RNP-A, RNP-68, Scl-70, Jo-1 and centromere B. The kit can include any 1, 2, 3, 4, 5, 6, 7, 8, 9, 10, or 11 additional antibodies in any combination.

In some embodiments, the kit includes at least one tube or other container with a known amount of the target of the at least one additional antibody. In some embodiments, the target of the at least one additional antibody is detectably labeled or attached to a matrix.

In some embodiments the kit includes a chimeric anti-dsDNA/chromatin antibody has a constant region from a human antibody and labeled secondary antibody specific for human antibodies (e.g., goat anti-human, rabbit anti-human, rat anti-human, etc.). In some embodiments, the kit includes at least one additional antibody with a different target specificity, wherein the at least one additional antibody is recognized by the same secondary antibody as the anti-dsDNA/chromatin antibody. In some embodiments, the at least one additional antibody is recognized by a different secondary antibody, e.g., labeled with a different label. In some embodiments, the at least one additional antibody has a constant region from a human antibody. The at least one additional antibody can either be a native antibody (e.g., derived from a human sample), a recombinantly produced antibody, or a chimeric antibody.

In some embodiments, the kit includes controls, e.g., a sample from an individual or pool of individuals known to carry anti-dsDNA/chromatin antibodies, or a sample from an individual or pool of individuals known to be negative for anti-dsDNA/chromatin antibody.

### VII. Examples

We obtained a mouse-human chimeric anti-dsDNA/chromatin IgG antibody for use as a calibrator in dsDNA and chromatin assays. The molecular biology work was performed by GenScript (Piscataway, N.J.).

The heavy and light chain variable regions of a mouse monoclonal antibody to dsDNA were sequenced and cloned into a human IgG constant region framework. After codon optimization, heavy and light chain co-expression was carried out initially in a transient HEK 293 system (vector pTGE5), and subsequently in a stable CHO expression system (vectors pGN, pcDNA3.1). Multiple clones were evaluated for chimeric antibody expression.

Antibody candidates were selected after initial testing for accelerated stability studies. The antibodies were recombinantly expressed and separated using Protein A column. Elution was carried out using Glycine-HCl Glycerol (highest yield and titer) or Glycyltyrosine. The separated antibodies were compared to conditioned media.

TABLE 1
Comparative separation results for dsDNA binding

| | IgG | | | | Corrected | |
|---|---|---|---|---|---|---|
| Elution | Concentration | Manual | dsDNA | dsDNA | dsDNA | |
| Method | (before dilution) | Dilution | RFI | IU/mL | IU/mL | dsDNA IU/mg |
| Glycine-HCl + | 0.147 mg/mL | 20 | 1312 | 50 | 1000 | 6803 |
| Glycerol | | | | | | |
| Glycine-HCl + | 0.202 mg/mL | 20 | 1830 | 69 | 1380 | 6832 |
| Glycerol | | | | | | |
| Glycine-HCl + | 0.351 mg/mL | 20 | 4024 | 142 | 2840 | 8091 |
| Glycerol | | | | | | |
| Glycyltyrosine | 0.230 mg/mL | 20 | 1812 | 69 | 1380 | 6000 |
| Conditioned | 0.745 mg/mL | 20 | 184 | 6 | 120 | 161 |
| Media, 48 hrs | | | | | | |
| Conditioned | 0.918 mg/mL | 20 | 191 | 6 | 120 | 131 |
| Media, 72 hrs | | | | | | |

TABLE 2
Comparative separation results for chromatin binding

| | | | | | Corrected | |
|---|---|---|---|---|---|---|
| Elution | IgG Concentration | Manual | Chromatin | Chromatin | Chromatin | Chromatin |
| Method | (before dilution) | Dilution | RFI | Al | Al | Al/mg |
| Glycine-HCl + | 0.147 mg/mL | 20 | 1705 | 10 | 206 | 1401 |
| Glycerol | | | | | | |
| Glycine-HCl + | 0.202 mg/mL | 20 | 2388 | 14 | 286 | 1416 |
| Glycerol | | | | | | |
| Glycine-HCl + | 0.351 mg/mL | 20 | 4430 | 27 | 546 | 1556 |
| Glycerol | | | | | | |
| Glycyltyrosine | 0.230 mg/mL | 20 | 2189 | 13 | 264 | 1148 |
| Conditioned | 0.745 mg/mL | 20 | 143 | 1 | 16 | 21 |
| Media, 48 hrs | | | | | | |
| Conditioned | 0.918 mg/mL | 20 | 167 | 1 | 20 | 22 |
| Media, 72 hrs | | | | | | |

Stability testing was carried out using commercial ANA kits (BioPlex™ 2200 from Bio-Rad) stored at 5° C. Glycine-Glycerol (GG) and Glycyltyrosine (GT) eluted antibodies were compared for stability.

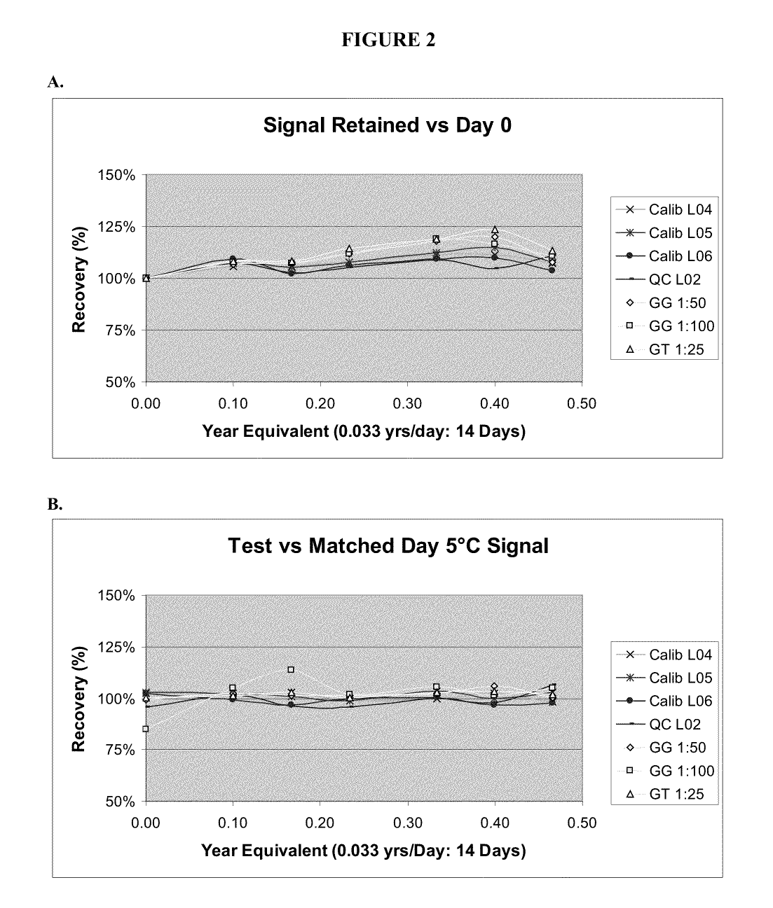

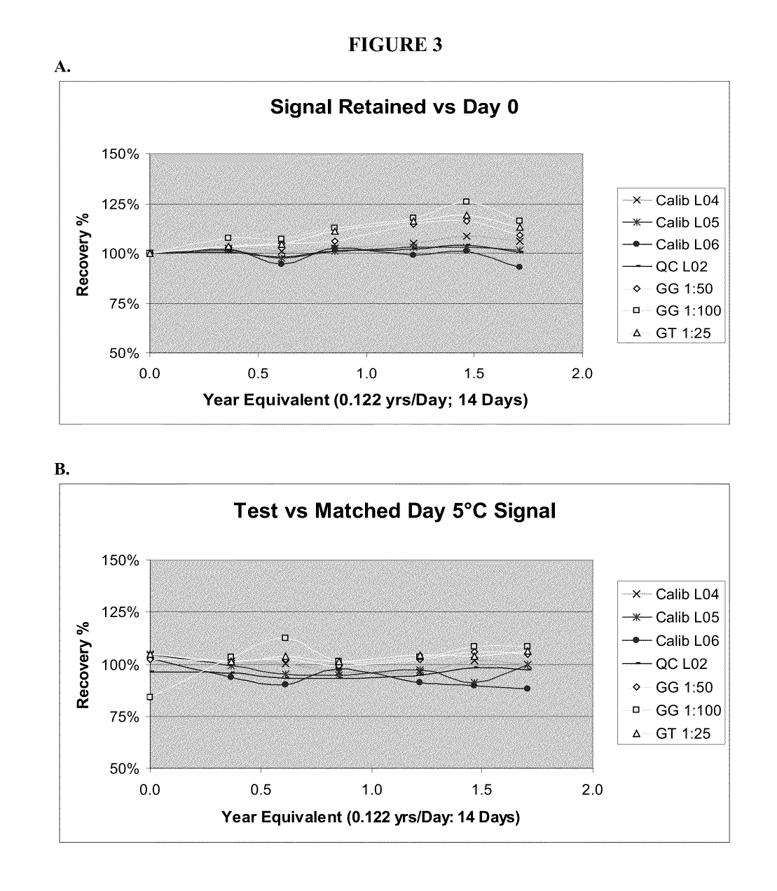

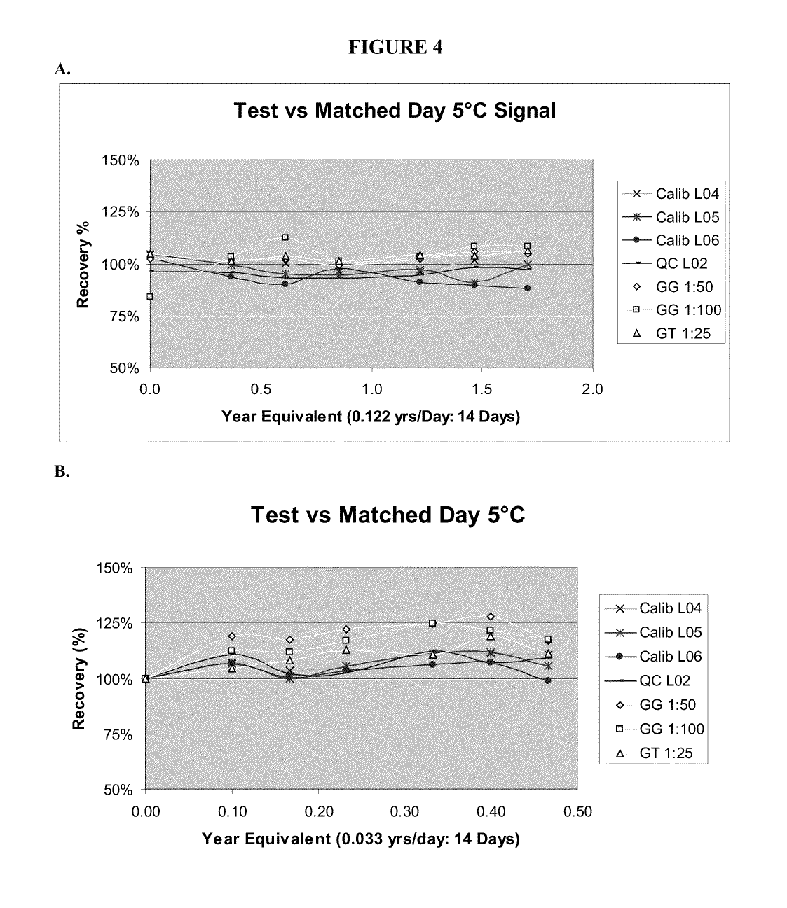

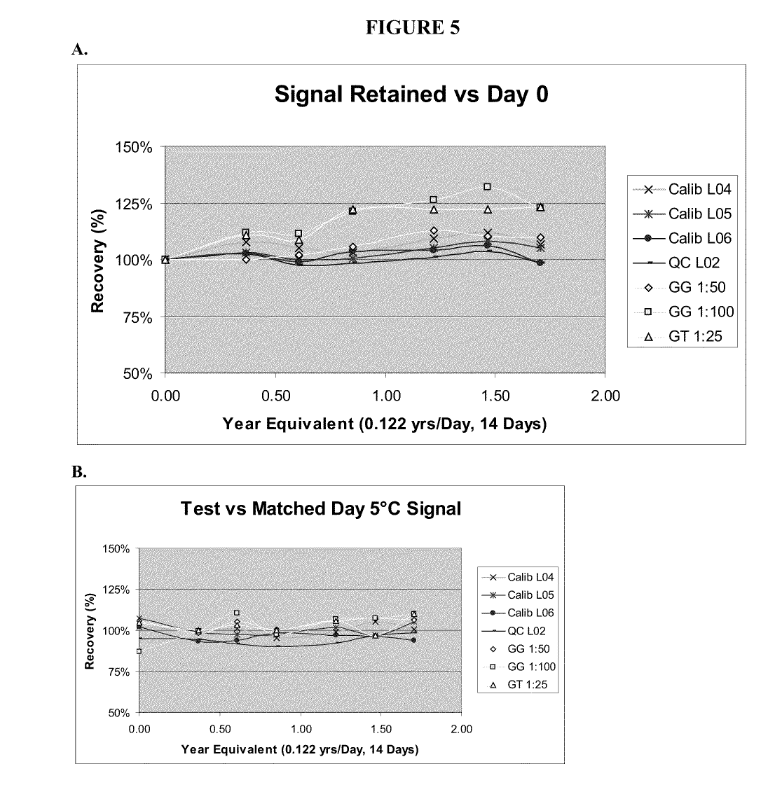

Dilutions of several antibody clones and conventional calibrator (control) antibodies were held at 5° C., 25° C., or 37° C. for up to 14 days (0.04, 0.47, 1.71 year-equivalents respectively) and tested periodically at 5° C. RFI (relative fluorescence intensity) increased slightly (˜10%) in all of the samples for the duration of the test across all temperatures. Signal comparisons between matched day 5° C. controls and elevated temperatures were usually within less than 10% of each other for both native (control) and chimeric antibodies (FIGS. 2 and 3). Similar results were obtained with the chromatin assays (FIGS. 4 and 5). Overall performance of the chimeric antibodies was very similar to the control antibodies.

It is understood that the examples and embodiments described herein are for illustrative purposes only and that various modifications or changes in light thereof will be suggested to persons skilled in the art and are to be included within the spirit and purview of this application and scope of the appended claims. All patents, patent applications, internet sources, and other published reference materials cited in this specification are incorporated herein by reference in their entireties. Any discrepancy between any reference material cited herein or any prior art in general and an explicit teaching of this specification is intended to be resolved in favor of the teaching in this specification. This includes any discrepancy between an art-understood definition of a word or phrase and a definition explicitly provided in this specification of the same word or phrase.

1. A chimeric antibody that specifically binds double stranded DNA (dsDNA) and chromatin, wherein at least part of the constant region of the chimeric antibody is derived from a human antibody.

2. The chimeric antibody of claim 1, wherein the chimeric antibody comprises complementarity determining regions (CDRs) derived from a non-human mammal.

3. (canceled)

4. The chimeric antibody of claim 1, wherein the chimeric antibody is stable at 5° C. for at least 5 months.

5. The chimeric antibody of claim 4, wherein the chimeric antibody is stable at 5° C. for at least 18 months.

6-7. (canceled)

8. The chimeric antibody of claim 1, wherein the chimeric antibody is bound to a labeled secondary antibody.

9. (canceled)

10. The chimeric antibody of claim 1, wherein the chimeric antibody specifically binds human dsDNA and chromatin.

11. (canceled)

12. The chimeric antibody of claim 1, wherein the chimeric antibody has a linear dilution profile within a range of 10-9000 relative fluorescence intensity (RFI) for dsDNA and within a range of 10-1500 RFI for chromatin.

13. A solution comprising the chimeric antibody of claim 1 and at least one additional antibody that specifically binds an antigen selected from the group consisting of: ribosomal protein, SS-A52, SS-A60, SS-B, Sm, Sm/ribonuclear protein (RNP), RNP-A, RNP-68, Scl-70, Jo-1, and centromere B.

14-15. (canceled)

16. A kit for determining the amount of a sample antibody that specifically binds dsDNA or chromatin, the kit comprising:
at least one container comprising a defined amount of the chimeric antibody of claim 1.

17-23. (canceled)

24. A method for generating a calibration curve of the chimeric antibody of claim 1, comprising:
contacting the chimeric antibody at a first known amount with dsDNA or chromatin in a first solution, detecting binding of the chimeric antibody to the dsDNA or chromatin, and assigning a first detection value to the first known amount of chimeric antibody;
contacting the chimeric antibody at a second known amount with dsDNA or chromatin in a second solution, wherein the dsDNA or chromatin is present at the same amount in the first and second solutions, detecting binding of the chimeric antibody to the dsDNA or chromatin, and assigning a second detection value to the second known amount of chimeric antibody,
thereby generating a calibration curve of the chimeric antibody.

25. The method of claim 24, further comprising repeating the steps of contacting, detecting, and assigning additional detection values for additional known amounts of chimeric antibody, wherein the dsDNA or chromatin is present at the same amount in each of the solutions.

26-27. (canceled)

28. The method of claim 24, wherein dsDNA is contacted, and the known amounts of chimeric antibody are in the range of 0.01 to 10 ug/mL.

29-30. (canceled)

31. A calibration curve generated according to the method of claim 24.

32. A method determining the amount of a sample antibody that specifically binds dsDNA or chromatin, comprising:
contacting the chimeric antibody of claim 1 at a first known amount with dsDNA or chromatin in a first solution, detecting binding of the chimeric antibody to the dsDNA or chromatin, and assigning a first detection value to the first known amount of chimeric antibody;
contacting the chimeric antibody at a second known amount with dsDNA or chromatin in a second solution, detecting binding of the chimeric antibody to the dsDNA or chromatin, and assigning a second detection value to the second known amount of chimeric antibody;
contacting the sample antibody with dsDNA or chromatin in a test solution, detecting binding of the sample antibody to the dsDNA or chromatin, and assigning a detection value to the sample antibody; and
comparing the detection value of the sample antibody to the first and second detection values, wherein the dsDNA or chromatin is present at the same amount in each of the solutions,
thereby determining the amount of sample antibody.

33. The method of claim 32, further comprising repeating the steps of contacting, detecting, and assigning additional detection values for additional known amounts of chimeric antibody, and comparing the detection value of the sample antibody to the additional detection values, wherein the dsDNA or chromatin is present at the same amount in each of the solutions.

34-38. (canceled)

39. The method of claim 32, wherein the sample antibody is obtained from a biological sample from a human.

40. The method of claim 39, further comprising determining whether the human has an autoimmune disease based on the amount of sample antibody.

41. The method of claim 40, wherein the autoimmune disease is selected from the group consisting of systemic lupus erythematosus, mixed connective tissue disease, Sjogren's syndrome, scleroderma, dermatomyositis, polymyositis, CREST syndrome, rheumatoid arthritis, juvenile arthritis, and Felty's syndrome.
